(19) United States
(12) Patent Application Publication
(10) Pub. No.: US 2007/0255401 A1
(43) Pub. Date: Nov. 1, 2007

(54) Design of Inlays With Intrinsic Diopter Power

(75) Inventor: Alan Lang, Long Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: ReVision Optics, Inc.

(21) Appl. No.: 11/381,056

(22) Filed: May 1, 2006

(51) Int. Cl. A61F 2/14

(52) U.S. Cl. 623/5.11; 623/901

(57) ABSTRACT

Described herein are designs and design methods for intracorneal inlays with intrinsic dioper power (i.e., index of refraction different from the surrounding cornea tissue). The designs and design methods achieve a desired refractive change by a combination of the intrinsic diopter power of the inlay and the physical shape of the inlay, which alters the shape of the anterior cornea surface.

### FIELD OF THE INVENTION

The field of the invention relates generally to corneal implants, and more particularly, to intracorneal inlays.

### BACKGROUND INFORMATION

As is well known, abnormalities in the human eye can lead to vision impairment. Some typical abnormalities include variations in the shape of the eye, which can lead to myopia (near-sightedness), hyperopia (far-sightedness) and astigmatism as well as variations in the tissue present throughout the eye, such as a reduction in the elasticity of the lens, which can lead to presbyopia. A variety of technologies have been developed to try and address these abnormalities, including corneal implants.

Corneal implants can correct vision impairment by altering the shape of the cornea. Corneal implants can be classified as an onlay and an inlay. An onlay is an implant that is placed over the cornea such that the outer layer of the cornea, e.g., the epithelium, can grow over and encompass the implant. An inlay is an implant that is surgically implanted into the cornea beneath a portion of the corneal tissue by, for example, cutting a flap in the cornea and inserting the inlay beneath the flap. Both inlays and outlays can alter the refractive power of the cornea by changing the shape of the anterior cornea, by having a different index of refraction than the cornea, or both. Since the cornea is the strongest refracting optical element in the human ocular system, altering the cornea's anterior surface is a particularly useful method for correcting vision impairments caused by refractive errors. Inlays are also useful for correcting other visual impairments including presbyopia.

### SUMMARY

Described herein are designs and design methods for intracorneal inlays with intrinsic dioper power (i.e., index of refraction different from the surrounding cornea tissue). The designs and design methods achieve a desired refractive change by a combination of the intrinsic diopter power of the inlay and the physical shape of the inlay, which alters the shape of the anterior cornea surface.

In an embodiment, a first-order inlay design method is provided, in which the refractive change provided by the intrinsic power and shape of the inlay is equivalent to treating the inlay as a contact lens in air.

In another embodiment, an increase in the refractive power of a patient's eye, e.g., to correct hyperopia, is provided by an inlay having a positive intrinsic power (i.e., index of refraction higher than that of the cornea) and/or an anterior surface having a higher curvature than the anterior corneal surface. In yet another embodiment, a decrease in refractive power, e.g., to correct myopia, is provided by an inlay having a negative intrinsic power (i.e., index of refraction lower than that of the cornea) and/or an anterior surface having a lower curvature than the anterior corneal surface.

The index of refraction of the inlay may be substantially uniform or non-uniform (i.e., vary within the inlay). In an embodiment, the index of refraction of an inlay is different at horizontal and vertical meridians to correct, e.g., astigmatism, by providing different diopter powers in the different meridians. In another embodiment, the index of refraction of the inlay varies along a radial direction to correct high-order aberrations including spherical aberration and coma, and/or to provide multiple optical zones. In another embodiment, the shape of an inlay is used to correct lower-order aberrations, e.g., spherical defocus, and the intrinsic power of the inlay is used to correct higher-order aberrations, e.g., astigmatism, spherical aberrations, and/or coma. In other embodiments, both the shape and the intrinsic power of the inlay may be used to correct higher-order aberrations.

In another embodiment, an initial inlay design is refined using an iterative ray-tracing procedure. In an exemplary embodiment, the shape and intrinsic diopter power of the inlay design are incorporated into a model of an eye. Ray tracing is then performed on the model eye to evaluate the inlay design and determine whether it achieves a targeted degree of correction. If not, then the shape of the inlay, intrinsic power of the inlay, or both are adjusted and the ray tracing is performed again on the model eye incorporating the inlay design. The process of adjusting parameters of the inlay design and performing ray tracing on the model eye is repeated until the inlay design achieves the targeted degree of correction or the design is optimized. In another embodiment, aberrations in a patients eyes are measured and incorporated into the model eye.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention not be limited to the details of the example embodiments.

### BRIEF DESCRIPTION OF THE FIGURES

FIG. 1 is a cross-sectional view of a cornea showing an intracorneal inlay implanted in the cornea according to an embodiment of the invention and the subsequent change in the cornea's anterior surface.

FIG. 2 is a cross-sectional view of the cornea showing a thickness profile of the inlay and a thickness profile on the anterior corneal surface.

FIG. 3 is a top-down view of the inlay.

### DETAILED DESCRIPTION

Described herein are designs and design methods for intracorneal inlays with intrinsic dioper power (i.e., index of refraction different from the surrounding cornea tissue). The designs and design methods achieve a desired refractive change by a combination of the intrinsic diopter power of the inlay and the physical shape of the inlay, which alters the shape of the anterior cornea surface.

FIG. 1 shows an example of an intracorneal inlay 10 implanted in a cornea. The intracorneal inlay may have a meniscus shape with an anterior surface 15 and a posterior surface 20. The intracorneal inlay 10 may be implanted in the cornea by cutting a flap into the cornea, lifting the flap, placing the inlay on the exposed area of the cornea's interior, and repositioning the flap over the inlay. The flap may be cut using a laser, e.g., a femtosecond lasers a mechanical keratome or manually by a ophthalmic surgeon. The inlay 10 is placed on a flap bed 30 in the cornea. Alternatively, a pocket or well (not shown) having side walls or barrier structures may be cut into the corneas, and the inlay placed between the side walls or barrier structures to prevent migration of the inlay in the cornea.

The implanted inlay 10 alters the shape of the anterior corneal surface, and therefore the refractive power of the cornea. In FIG. 1, the pre-operative anterior corneal surface is represented by dashed line 35 and the post-operative anterior corneal surface induced by the inlay is represented by solid line 40.

A method for designing an intracorneal inlay will now be described with reference to FIG. 1. A first step is to determine the change in refractive power needed to correct a patient's vision. The desired refractive change can be measured by an optometrist or ophthalmic surgeon. Let the refractive power change at the corneal optical plane be ΔK.

For intracorneal inlay designs, it is sufficient to use paraxial optics for a first-order design. Refinements to the first-order design using ray-tracing techniques are given below. The refractive power change at the corneal optical plane ΔK induced by the inlay may be written as:
ΔK=(n_{c}−1)(cₚₒₛₜₒₚ−cₚᵣₑₒₚ)+P_{inlay} Equation 1
Where n_{c} is the index of refraction of the cornea, cₚₒₛₜₒₚ is the post-operative curvature of the anterior corneal surface, cₚᵣₑₒₚ is the pre-operative curvature of the anterior corneal surface (i.e., before implantation of the inlay), and P_{inlay} is the intrinsic refractive power of the inlay. Using paraxial approximation, P_{inlay} may be written as:
P_{inlay}=(n_{I}−n_{c})(cₐₙₜ−cₚₒₛₜ) Equation 2
where n_{I} is the index of refraction of the inlay material, cₐₙₜ is the curvature of the inlay's anterior surface, and cₚₒₛₜ is the curvature of the inlay's posterior surface.

Note that if n_{I}=n_{c}, then the intrinsic power of the inlay is zero, and the change in the refractive power in Equation 1 is due solely to the change in the shape of the anterior corneal surface induced by the shape of the inlay.

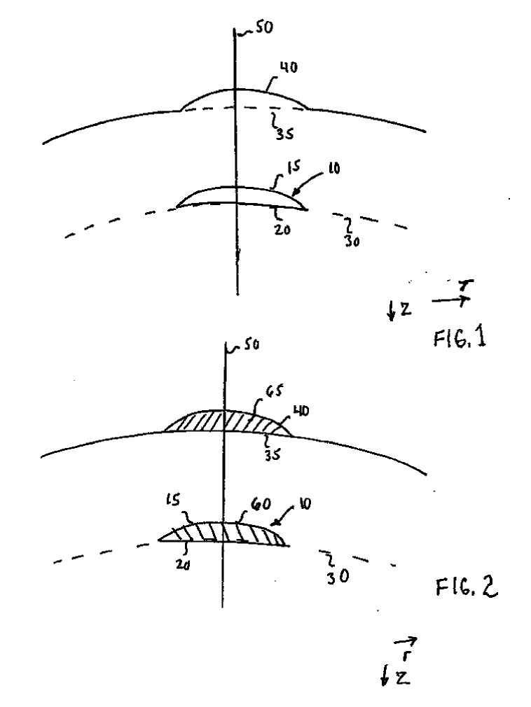

Biomechanically, the inlay implanted in the cornea alters the curvature of the anterior corneal surface. The effects of the inlay shape on the curvature of the anterior corneal surface can be modeled by assuming that the axial thickness profile of the inlay is translated to the anterior corneal surface through the intervening flap. Based on this assumption, the axial thickness profile of the inlay equals the axial thickness profile between the post-operative and pre-operative anterior corneal surfaces. This assumption is illustrated in FIG. 2, in which the thickness profile 60 of the inlay is translated to the anterior corneal surface as the thickness profile 65 between the post-operative and pre-operative anterior corneal surfaces. An optical axis 50 is shown in FIG. 2. Additional details on the assumption of equivalent thickness profiles can be found in U.S. patent application Ser. No. 11/293,644, titled “Design Of Intracorneal Inlays,” filed on Dec. 1, 2005, the entirety of which is incorporated herein by reference.

The saggital height of an axial symmetric surface as a function of radial location r can be expressed as Z(r). Z(r) is a finction of curvature c. The above assumption of equal profiles implies that:
Zₚᵣₑₒₚ(r,cₚᵣₑₒₚ)−Zₚₒₛₜₒₚ(r,cₚₒₛₜₒₚ)=Z_{Ipost}(r,cₚₒₛₜ₎₋Z_{Iant}(r,cₐₙₜ) Equation 3
where the subscript “preop” indicates the pre-operative anterior corneal surface, “postop” indicates the post-operative anterior corneal surface, “Ipost” indicates the posterior surface of the inlay, and “Iant” indicates the anterior surface of the inlay. The z direction, radial r direction, and optical axis 50 are shown in FIGS. 1 and 2.

With the above set of equations, all inlay design method according to an embodiment comprises fixing some of the parameters in Equations 1-3 and solving for the other parameters. For example, the parameters ΔK, cₚᵣₑₒₚ, and n_{c} are generally known. The desired refractive change ΔK and pre-operative anterior corneal surface cₚᵣₑₒₚ can be measured by, e.g., an optometrist or ophthalmic surgeon. The index of fraction n_{c} of the cornea is approximately equal to 1.376. As for the remaining parameters cₚₒₛₜₒₚ, cₚₒₛₜ, cₐₙₜ, n_{I} and P_{inlay}, an inlay may be designed by fixing two of these parameters and solving for the other three parameters. For example, the posterior curvature cₚₒₛₜ of the inlay may be shaped to approximate the geometry of the flap bed, and therefore be fixed. Further, the index of refraction n_{I} of the inlay may be fixed by the inlay material. With cₚₒₛₜ and n_{I} fixed, Equations 1-3 can be used to solve for the three unknown parameters cₚₒₛₜₒₚ, cₐₙₜ, and P_{inlay}. After the unknown parameters are solved, the resulting design for the intracorneal inlay with intrinsic power can be specified by the parameters cₐₙₜ, cₚₒₛₜ, and n_{I}, where cₐₙₜ and cₚₒₛₜ define the shape of the inlay and n_{I} defines the index of refraction of the inlay. The inlay design is also specified by the center thickness of the inlay, which may be chosen based on considerations of desired inlay diameter, and biophysiological responses of the cornea to inlay thickness.

For a first-order design, the surface parameter Z(r) may be approximated using the paraxial approximation and assuming small r, in which case Z(r)≈cr²/2. Using this approximation, Equation 3 reduces to:
cₚᵣₑₒₚ−cₚₒₛₜₒₚ=cₚₒₛₜ−cₐₙₜ Equation 4

Substituting Equations 1, 2 and 5 yields:
ΔK=(cₐₙₜ−cₚₒₛₜ)(n_{I}−1) Equation 5

Equation 5 equals the refractive power of the inlay in air, which is equivalent to treating the inlay as a contact lens in air. Equation 5 is useful in determining a design for an inlay with intrinsic power. For example, the anterior curvature cₐₙₜ of an inlay can be readily calculated if the other parameters are known by simply measuring the inlay's diopter power in air. In this example, n_{I} may be fixed by the inlay material and cₚₒₛₜ may be fixed by the geometry of the flap bed.

The solution for a general form of Z(r) may be nonlinear. For example, the surface parameter Z(r) may be expressed in the form:

$$Z ( r ) = \frac{{cr}^{2}}{1 + \sqrt{1 - ( 1 + k ) {( cr )}^{2}}} + \sum {a}_{n} {r}^{2 n} \qquad Equation 6$$

where c is the curvature of the surface, k is a conic constant, and aₙ are higher order aspheric constants. For a spherical surface, the constants k and aₙ are zero. A typical human cornea may be approximated by k=−0.16 and aₙ=0. The constants k and aₙ may be used in more advanced designs to correct or mitigate higher order aberrations.

The refractive change ΔK induced by the inlay is provided by a combination of the power change due to the shape of the inlay (e.g., (n_{c}−1)(cₐₙₜ−cₚₒₛₜ)) and the intrinsic power of the inlay (e.g., (n_{I}−n_{c})(cₐₙₜ−cₚₒₛₜ)). Thus, this design method allows the diopter power of the patient's eye to be adjusted by two mechanisms: change in the shape of the anterior corneal surface induced by the shape of the inlay and the intrinsic diopter power of the inlay. To adjust the intrinsic power of the inlay, the index of refraction n_{I} of the inlay may be adjusted in the range of 1.33 to 1.55 by selecting different materials for the inlay including, but not limited to, Lidofilcon A, Poly-HEMA, polysulfone, silicone hydrogel, and the like.

For example, an increase in refractive power, e.g., to correct hyperopia, may be achieved by an increase in the curvature of the anterior corneal surface and/or a positive intrinsic power of the inlay. For example, the inlay may be designed with a higher surface curvature than the anterior corneal surface and/or a positive intrinsic power (i.e., index of refraction higher than n_{c}=1.376) to increase the refractive power of the patient's eye.

A decrease in refractive power, e.g., to correct myopia, may be achieved by a decrease in the curvature of the anterior corneal surface and/or a negative intrinsic power of the inlay. For example, the inlay may be designed with a smaller curvature than the anterior corneal surface and/or a negative intrinsic power (i.e., index of refraction lower than n_{c}=1.376).

For large refractive changes, e.g., to correct severe hyperopia, the cornea may adversely react to large changes in curvature, e.g., due to stress in the cornea, which may lead to complications. Therefore, the curvature of the inlay may be limited by the amount of change in curvature that the cornea can tolerate. In an embodiment, the anterior curvature of the inlay is limited to a range that the cornea can tolerate with the remaining refractive change being achieved by the intrinsic power of the inlay.

A design method according to an embodiment employs ray-tracing techniques to refine an inlay design. Ray tracing is a well known optic design technology that simulates the path of light rays through an optical system to determine whether the optical system achieves desired optical results. Since the human eye is an optical system, the human eye can be modeled by a finite physical model and evaluated using ray-tracing techniques to determine whether a desired image quality is achieved on the retina. An example of a finite model eye can be found in H. -L. Liou and N. A. Brennan, “Anatomically accurate, finite model eye for optical modeling”, Journal of the Optical Society of America, A/Vol. 14, No. 8, Aug. 1997. The model eye may include parameters for modeling optical elements of the eye including the curvature of the anterior corneal surface, the crystalline lens, etc.

Aberrations of a particular patient's eye may be incorporated into a model eye used for ray tracing. For example, the shape of the patient's anterior corneal surface can be measured based on a photograph of the anterior corneal surface or by reflecting rings off the anterior corneal surface, and determining the shape of the surface based on deformations in the reflected rings. Wavefront aberrometers may be used to measure internal aberrations in the eye. These measurements can then be incorporated into the model eye. Some of the parameters for the model eye may be based on measurements of the patient's eye, while other parameters may be based on an average representative eye. Thus, a model eye may be modified to model the eye of a particular patient, and therefore incorporate aberrations of the patient's eye.

Rather than customizing a human eye model for a particular patient, a human eye model may be chosen from a set of human eye models. For example, different human eye models may correspond to different ranges of targeted refractive changes, and the human eye model may be chosen for a particular patient based on the targeted refractive change for that patient.

The effects of the inlay can be incorporated into the model eye using Equations 1 and 3. For example, the effects of the inlay on the shape of the anterior corneal surface can be modeled based on the equivalent thickness profile assumption of Equation 3. In this example, the thickness profile of the inlay is translated one-to-one to the anterior corneal surface. In another embodiment, the equivalent thickness profile assumption may be part of a more complicated model of the biomechanical response of the anterior corneal surface to the inlay that also takes into account effects of the flap over the inlay.

After the inlay has been incorporated into the model eye, the effectiveness of the inlay design in correcting vision can be evaluated by performing ray tracing on the model eye, and evaluating the quality of the retinal image using an optical image quality metric. An example of an optical image quality metric is the modulation transfer function, which measures the effectiveness of transferring the contrast of the object into the contrast of the image. Examples of image quality metrics based on the modulation transfer function can be found in “Introduction to the Optical Transfer Function”, Williams and Becklund, Wiley & Sons, 2002.

In an embodiment, an inlay is designed by an iterative process in which one or more parameters of the inlay are adjusted and the inlay design is evaluated by ray tracing a model eye incorporating the inlay. This iterative process is repeated until the inlay design achieves a targeted degree of correction or the design is optimized. In an embodiment, the inlay shape may be held fixed, and the index of refraction n_{I} of the inlay may be adjusted until the targeted degree of correction is achieved using ray tracing. In another embodiment both the inlay shape and index of refraction n₁ may be adjusted.

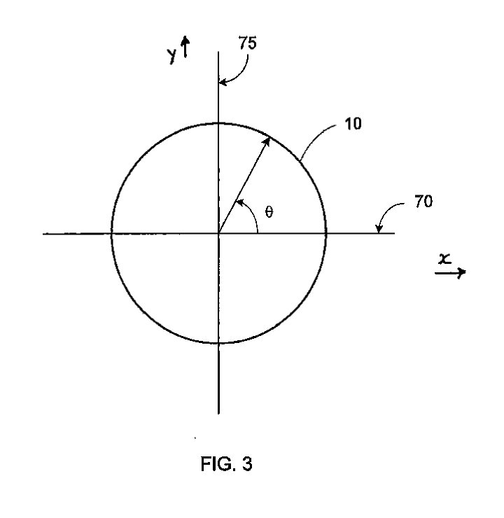

The index of refraction n_{I} may vary within the inlay to correct higher order aberrations, e.g., spherical aberrations. For example, the index of refraction n_{I} may vary with radial location r, asimuthal angle θ, or both. The asimuthal angle θ is in the plane containing the diameter of the inlay and is shown in the top-down view of the inlay in FIG. 3. In this embodiment, the intrinsic power P_{inlay} of the inlay may be written as:
P_{inlay}=(n_{I}(r,θ)−n_{c})(cₐₙₜ−cₚₒₛₜ) Equation 7
where n₁ is a function or radial location r and asimuthal angle θ. In is embodiment, the index of refraction n_{I} varies in a cylindrical coordinate system. The index of refraction n_{I} may also vary based on other coordinate systems. The inlay according to Equation 7 may be designed using the ray-tracing design method above based on Equations 1, 3, and 7. The inlay shape may be fixed with the index finction (n_{I}(r, θ)) being adjusted until a desired degree of correction is achieved. Alternatively, both the inlay shape and index function may be adjusted. In another embodiment, spherical defocus of a patient's eye may be corrected by a spherical shape of the inlay with higher order aberrations, e.g., astigmatism, being corrected by variations in the index of refraction n_{I} of the inlay.

The index of refraction n_{I} may be varied within the inlay in a number of ways. For example, the index of refraction n_{I} may be varied within a polymer inlay by using phase separation techniques, light, heat, electricity, or chemical gradients to create different index of refraction zones during the atucal polymerization process. Another method is to join materials with different index of refractions to form a composite material and fabricating the inlay from the composite material.

Astigmatism occurs when irregularities in the shape of the corneal causes the eye to have different focal points in the horizontal and vertical meridians. As a result, the eye cannot focus simultaneous in both meridians. To correct astigmatism, a corrective lens may have a higher diopter power in one meridian than the other meridian to align both focal points on the retina. Transition regions between the vertical and horizontal meridians may vary between these two powers. In an embodiment, the index of refraction n_{I} of the inlay is varied as a finction of the asimuthal angle θ to provide different diopter powers in the two meridians. For example, the index of refraction n_{I} may be higher in one meridian than the other meridian to give the inlay a higher diopter power in one meridian than the other meridian. FIG. 3 shows an example of a horizontal meridian 70 and a vertical meridian 75. As a example, correction of a particular patient with both mean spherical error and astigmatism may require a power of +1 diopter in the vertical meridian and a power of +2 diopters in the horizontal meridian. In this example, the index of refraction n_{I} of the inlay may be higher in the horizontal meridian than the vertical meridian to achieve the desired diopter power in each meridian. The +1 diopter and +2 diopter in the separate meridians will alter the mean refractive power by 1.5 diopters and correct 1 diopter of astigmatism. Astigmatism may also be corrected by a combination of inlay shape and variation in the index of refraction n_{I} of the inlay. For example, the inlay may have both a higher curvature and a higher index of refraction n_{I} in the meridian requiring higher diopter power.

To describe a surface with different curvatures in two separate meridians, the surface parameter Z(r) may be written in the form:

$$Z ( r ) = \frac{{c}_{x} {x}^{2} + {c}_{y} {y}^{2}}{1 + \sqrt{1 - ( 1 + {k}_{x} ) {c}_{x}^{2} {x}^{2} - ( 1 + {k}_{y} ) {c}_{y}^{2} {y}^{2}}} + \sum {a}_{n} {P}_{n} ( x , y ) \qquad Equation 8$$

where cₓ and kₓ are the curvature and conic constant for the meridian in the x direction, c_{y} and k_{y} are the curvature and conic constant for the meridian in the y direction, and aₙ are coefficients of a general polynomial expansion Pₙ in orders of x and y. Examples of the x and y directions are shown in FIG. 3. The different curvatures and conic constants in the x and y directions allow for different curvatures in the two meridians and for the correction of astigmatism by altering the anterior corneal surface in the two separate meridians.

The index of refraction n_{I} of the inlay may be varied along the radial direction r to correct high-order aberrations including spherical aberrations, coma, and trefoil. The index of refraction n_{I} may also be varied along the radial direction r to provide a multifocal inlay with multiple optical zones.

A variety of solutions are possible, depending on the what parameters are assumed fixed. In the case of a fixed index of refraction (e.g., fixed function n_{I}(r, θ)), the optimal and constant cₐₙₜ can be found by optimizing using the ray-traced based criteria above. Alternatively, given a targeted degree of astigmatic or aberration correction is fixed, and the ray tracing is iterated until the optimal index finction (n_{I}(r, θ)) is found.

Additionally, the ray-tracing process may show that a non-spherical shape to the anterior inlay's surface may be required.

In the foregoing specification, the invention has been described with reference to specific embodiments thereof. It will, however, be evident that various modifications and changes may be made thereto without departing from the broader spirit and scope of the invention. As another example, each feature of one embodiment can be mixed and matched with other features shown in other embodiments. As yet another example, the order of steps of method embodiments may be changed. Features and processes known to those of ordinary skill may similarly be incorporated as desired. Additionally and obviously, features may be added or subtracted as desired. Accordingly, the invention is not to be restricted except in light of the attached claims and their equivalents.

1. A method for designing an intracorneal inlay, comprising:
determining a desired refractive power change needed to correct a patient's vision;
determining a combination of an inlay shape and an intrinsic diopter power that achieves the desired refracted power change; and
shaping the inlay based on the determined inlay shape.

2. The method of claim 1, further comprising varying the index of refraction of the inlay within the inlay.

3. The method of claim 2, further comprising varying the index of refraction of the inlay along an asimuthal angle θ.

4. The method of claim 2, further comprising varying the index of refraction of the inlay along a radial direction.

5. The method of claim 1, wherein the index of refraction of the inlay is substantially uniform.

6. The method of claim 1, wherein the index of refraction of the inlay is higher than the index of refraction of a cornea.

7. The method of claim 6, wherein a curvature of an anterior surface of the inlay is higher than a curvature of the anterior corneal surface of the patient's eye.

8. The method of claim 1, wherein the index of refraction of the inlay is lower than the index of refraction of a cornea.

9. The method of claim 8, wherein a curvature of an anterior surface of the inlay is lower than a curvature of the anterior corneal surface of the patient's eye.

10. The method of claim 1, wherein the inlay has vertical and horizontal meridians and the index of refraction of the inlay is higher in one of the meridians than the other meridian.

11. The method of claim 10, wherein an anterior surface of the inlay has different curvatures in the two meridians.

12. The method of claim 1, wherein the inlay has vertical and horizontal meridians and an anterior surface of the inlay has different curvatures in the two meridians.

13. The method of claim 1, further comprising:
cutting a flap into one of the patient's cornea;
lifting the flap to expose an interior of the patient's cornea;
placing the inlay in the interior of the patient's cornea; and
repositioning the flap over the inlay.

14. The method of claim 1, further comprising:
cutting a pocket in the interior of one of the patient's cornea; and
placing the inlay in the pocket.

15. A method for designing an intracorneal inlay, comprising:
determining a desired refractive power change needed to correct a patient's vision;
determining a combination of an inlay shape and an intrinsic diopter power that achieves the desired refracted power change; and
choosing an index of refraction for the inlay based on the determined intrinsic diopter power.

16. The method of claim 15, further comprising varying the index of refraction of the inlay within the inlay.

17. The method of claim 16, further comprising varying the index of refraction of the inlay along an asimuthal angle θ.

18. The method of claim 16, further comprising varying the index of refraction of the inlay along a radial direction.

19. The method of claim 15, wherein the index of refraction of the inlay is substantially uniform.

20. The method of claim 15, wherein the index of refraction of the inlay is higher than the index of refraction of a cornea.

21. The method of claim 20, wherein a curvature of an anterior surface of the inlay is higher than a curvature of the anterior corneal surface of the patient's eye.

22. The method of claim 15, wherein the index of refraction of the inlay is lower than the index of refraction of a cornea.

23. The method of claim 22, wherein a curvature of an anterior surface of the inlay is lower than a curvature of the anterior corneal surface of the patient's eye.

24. The method of claim 15, wherein the inlay has vertical and horizontal meridians and the index of refraction of the inlay is higher in one of the meridians than the other meridian.

25. The method of claim 24, wherein an anterior surface of the inlay has different curvatures in the two meridians.

26. The method of claim 15, wherein the inlay has vertical and horizontal meridians and an anterior surface of the inlay has different curvatures in the two meridians.

27. The method of claim 15, further comprising:
cutting a flap into one of the patient's cornea;
lifting the flap to expose an interior of the patient's cornea;
placing the inlay in the interior of the patient's cornea; and
repositioning the flap over the inlay.

28. The method of claim 15, further comprising:
cutting a pocket in the interior of one of the patient's cornea; and
placing the inlay in the pocket.

29. A method for designing an intracorneal inlay, comprising:
(a) determining a desired refractive power change needed to correct a patient's vision;
(b) determining a combination of an inlay shape and an intrinsic diopter power for an inlay design;
(c) incorporating the inlay design into a model eye;
(d) performing ray tracing on the model eye incorporating the inlay design to determine whether a targeted degree of correction is achieved by the inlay design;
(e) if the targeted degree of correction is not achieved, adjusting the shape of the inlay design, the intrinsic diopter power of the inlay design, or both; and
(f) repeating steps (d) and (e) until the inlay design achieves the targeted degree of correction.

30. The method of claim 29, further comprising varying the index of refraction of the inlay design.

31. The method of claim 30, further comprising varying the index of refraction of the inlay design along an asimuthal angle θ.

32. The method of claim 30, further comprising varying the index of refraction of the inlay design along a radial direction.

33. The method of claim 29, wherein the index of refraction of the inlay design is substantially uniform.

34. The method of claim 29, wherein the index of refraction of the inlay design is higher than the index of refraction of a cornea.

35. The method of claim 34, wherein a curvature of an anterior surface of the inlay design is higher than a curvature of the anterior corneal surface of the patient's eye.

36. The method of claim 29, wherein the index of refraction of the inlay design is lower than the index of refraction of a cornea.

37. The method of claim 36, wherein a curvature of an anterior surface of the inlay design is lower than a curvature of the anterior corneal surface of the patient's eye.

38. The method of claim 29, wherein the inlay design has vertical and horizontal meridians and the index of refraction of the inlay is higher in one of the meridians than the other meridian.

39. The method of claim 38, wherein an anterior surface of the inlay has different curvatures in the two meridians.

40. The method of claim 29, wherein the inlay has vertical and horizontal meridians and an anterior surface of the inlay has different curvatures in the two meridians.

41. The method of claim 29, further comprising measuring a parameter of a patient's eye and incorporating the measured parameter into the model eye.

42. The method of claim 41, wherein the measured parameter is the shape of an anterior corneal surface of the patient's eye.

43. The method of claim 29, wherein the combination of the shape and intrinsic diopter power of the inlay design is determined based on the following equation:
K=(cₐₙₜ−cₚₒₛₜ)(n_{I}−1)
where ΔK is the desired refractive change, cₐₙₜis an anterior surface curvature of the inlay design, cₐₙₜ is a posterior surface curvature of the inlay design, and n_{I} is an index of refraction of the inlay design.
